(19) United States
(12) Patent Application Publication
(10) Pub. No.: US 2020/0268639 A1
(43) Pub. Date: Aug. 27, 2020

(54) OILY SOLID COSMETIC

(71) Applicant: TOKIWA CORPORATION, Nakatsugawa-shi (JP)

(72) Inventor: Kenji KOKAI, Saitama (JP)

(21) Appl. No.: 16/793,071

(22) Filed: Feb. 18, 2020

(30) Foreign Application Priority Data
Feb. 27, 2019 (JP) 2019-034354

(51) Int. Cl. A61K 8/92; A61K 8/891; A61K 8/25; A61K 8/29; A61K 8/37; A61K 8/26; A61K 8/19; A61Q 1/08; A61Q 1/10; A61K 8/02

(52) U.S. Cl. CPC A61K 8/92; A61K 8/891; A61K 8/25; A61K 8/29; A61K 8/0216; A61K 8/26; A61K 8/19; A61Q 1/08; A61Q 1/10; A61K 8/375

(57) ABSTRACT

To provide an oily solid cosmetic having sufficient impact resistance and favorable use performance and use feeling (it is easy to scoop an appropriate amount by a finger and the cosmetic is easy to spread and has excellent tight adhesion, a makeup film with less pearl irregularity, and a moist finish) while the solid cosmetic can give sufficient elasticity feeling when touched by a finger. This oily solid cosmetic includes: a powder ingredient in an amount of 40 to 70 mass %, the powder ingredient containing sparkling powder; (A) silicone wax with a melting point of 25 to 55° C.; and (B) a crosslinked silicone elastomer.

### TECHNICAL FIELD

The present invention relates to an oily solid cosmetic.

### BACKGROUND ART

Widely used are solid powder cosmetics, in which powder ingredients are compacted, as eye shadow, foundation, and cheek makeup cosmetics, etc. The powder cosmetics are characterized by dry touch because they are easy to spread and powdery. However, the powder ingredients are unlikely to adhere tightly to the skin and it is also hard to produce a moist finish. In addition, sparkling powder such as a pearl pigment is unlikely to adhere tightly to the skin because the particle size is much larger than that of regular color pigments. Thus, it is difficult for powder cosmetics, in which sparkling powder is abundantly blended, to form a pearl irregularity-less, uniform makeup film.

Recently, research has been in progress on cosmetics, in an intermediate state (funicular state) between powder cosmetics and oily cosmetics, that has both the powder cosmetic features and the oily cosmetic features such as moist feeling, adhesion, and cosmetic durability. During the research, solid cosmetics have been developed that give, as a new touch not obtained by conventional powder cosmetics or oily cosmetics, flexibility and elasticity feelings upon a finger touch (see, for example the following Patent Literatures 1 and 2). Such solid cosmetics tend to have poor impact resistance when the elasticity is sought. Thus, it has been considered to establish both impact resistance and elasticity by adjusting oily ingredients and/or blending a polymer (see, for example, the following Patent Literatures 3 and 4).

### CITATION LIST

### Patent Literature

Patent Literature 1: JP 2006-1883 A

Patent Literature 2: JP 2005-314369 A

Patent Literature 3: JP 2014-218469 A

Patent Literature 4: JP 2016-190837 A

### SUMMARY OF INVENTION

### Technical Problem

Unfortunately, the above solid cosmetics are not yet enough to obtain a moist finish. Here, when a finger is used to apply a cosmetic, consumers can feel cosmetic elasticity more. However, the above solid cosmetics are hard to scoop an appropriate amount because they are scooped in a crumbled state during finger scooping.

Here, it is an object of the present invention to provide an oily solid cosmetic having sufficient impact resistance and favorable use performance and use feeling (it is easy to scoop an appropriate amount by a finger and the cosmetic is easy to spread and has excellent tight adhesion, a makeup film with less pearl irregularity, and a moist finish) while the solid cosmetic can give sufficient elasticity feeling when touched by a finger.

### Solution to Problem

To solve the above problem, the present inventors have conducted intensive research and, as a result, have found that in an oily solid cosmetic containing a specific amount of a powder ingredient, silicone wax with a particular melting point and a crosslinked silicone elastomer are blended in combination to provide a pressed molded product or compacted body in which the above use performance and use feeling can be obtained while both elasticity and impact resistance are established even when sparkling powder is abundantly included as a powder ingredient.

Specifically, the invention provides an oily solid cosmetic including: a powder ingredient in an amount of 40 to 70 mass %, the powder ingredient including sparkling powder; (A) silicone wax with a melting point of 25 to 55° C.; and (B) a crosslinked silicone elastomer.

According to the oily solid cosmetic of the present invention, a compact can be formed that has sufficient impact resistance and favorable use performance and use feeling (it is easy to scoop an appropriate amount by a finger and the cosmetic is easy to spread and has excellent tight adhesion, a makeup film with less pearl irregularity, and a moist finish) while the sufficient elasticity can be felt by a finger.

In the oily solid cosmetic of the invention, the content of the sparkling powder may be 30 mass % or higher with reference to the total amount of the oily solid cosmetic. Even when the sparkling powder is abundantly included as the powder ingredient, the above effects can be obtained.

From the viewpoint of further increasing the elasticity, finger scooping, and impact resistance, the oily solid cosmetic of the invention preferably includes, as the above ingredient (A), silicone wax with a melting point of 45° C. or lower and a hardness at 25° C. of 0.5 to 5.0 N as measured with a rheometer (under measurement conditions in which the tip shape of a pressure-sensing shaft is like a disk with a diameter of 5 mm, the penetration speed is 2 cm/min, and the penetration depth is 3 mm).

In addition, from the viewpoint of giving elasticity in the oily solid cosmetic of the present invention, a mixture containing low-viscosity oil with a viscosity at 25° C. of 5 to 50 mPa·s and the crosslinked silicone elastomer dispersed in the low-viscosity oil is blended in an amount of 20 to 45 mass % with reference to the total amount of the oily solid cosmetic and the mixture preferably has a hardness at 25° C. of 0.05 to 0.5 N as measured with a rheometer (under measurement conditions in which the tip shape of a pressure-sensing shaft is like a sphere with a diameter of 10 mm, the penetration speed is 6 cm/min, and the penetration depth is 10 mm).

The oily solid cosmetic of the invention may be a compact.

### Advantageous Effects of Invention

The invention can provide an oily solid cosmetic having sufficient impact resistance and favorable use performance and use feeling (it is easy to scoop an appropriate amount by a finger and the cosmetic is easy to spread and has excellent tight adhesion, a makeup film with less pearl irregularity, and a moist finish) while the solid cosmetic can give sufficient elasticity feeling when touched by a finger.

### Description of Embodiments

An oily solid cosmetic according to an embodiment of the invention includes a powder ingredient in an amount of 40 to 70 mass %; (A) silicone wax with a melting point of 25 to 55° C. (hereinafter, sometimes referred to as ingredient (A)); and (B) a crosslinked silicone elastomer (hereinafter, sometimes referred to as ingredient (B)).

(Silicone Wax with Melting Point of 25 to 55° C.)

As the silicone wax with a melting point of 25 to 55° C., a silicone-modified acrylic polymer, alkyl-modified silicone, or aliphatic alcohol-modified silicone may be used. Among them, alkyl-modified silicone is preferable from the viewpoint of silicone elastomer dispersion property.

The silicone-modified acrylic polymer is a polysiloxane-containing acrylic polymer and examples include a (acrylate/stearyl acrylate/dimethicone methacrylate) copolymer or a (acrylate/behenyl acrylate/dimethicone methacrylate) copolymer. Commercially available products such as KP561P (trade name, manufactured by Shin-Etsu Chemical Co., Ltd,) and KP562P (trade name, manufactured by Shin-Etsu Chemical Co., Ltd.) are applicable to them.

The alkyl-modified silicone is an alkyl group-containing silicone. The alkyl group is preferably a C₁₂₋₅₀ linear or branched chain and more preferably a C₁₈₋₄₀ linear or branched chain, and may be substituted at any of a side chain, one end, or both ends of the polysiloxane backbone. Examples of such alkyl-modified silicone include stearyl dimethicone, alkyl (C₂₆₋₂₈) dimethicone, or cetearyl methicone. Here, a commercially available product such as 2503 Cosmetic WAX (trade name, manufactured by Dow Corning Toray Co., Ltd.), BELSIL SDM 5055VP (trade name, manufactured by Wacker Asahikasei Silicone Co., Ltd.), BELSIL CDM 3526VP (trade name, manufactured by Wacker Asahikasei Silicone Co., Ltd.), or SF1632 (trade name, manufactured by Momentive Performance Materials, Inc.) may be used.

The aliphatic alcohol-modified silicone is an alkoxy group-containing silicone. The alkoxy group is preferably a C₁₂₋₅₀ linear or branched chain and more preferably a C₁₈₋₄₀ linear or branched chain, and may be substituted at any of a side chain, one end, or both ends of the polysiloxane backbone. Examples of such aliphatic alcohol-modified silicone include a (stearoxy methicone/dimethicone) copolymer, stearoxy dimethicone, or behenoxy dimethicone. Here, a commercially available product such as KF7002 (trade name, manufactured by Shin-Etsu Chemical Co., Ltd.), BELSIL W3230 (trade name, manufactured by Wacker Asahikasei Silicone Co., Ltd.), ABIL Wax 2434 (trade name, Evonik Japan Co., Ltd.), or ABIL Wax 2440 (trade name, Evonik Japan Co., Ltd.) may be used.

The oily solid cosmetic of the embodiment preferably includes silicone wax with a melting point of 25 to 45° from the viewpoint of making easier to scoop an appropriate amount by a finger while securing sufficient impact resistance.

Note that as used herein, the melting point of silicone wax means a value measured by the following protocol.

About 5 mg of each sample is weighed and then put into an aluminum sample pan. An aluminum cover is attached to this pan, which is then installed in a differential scanning calorimeter “DSC7020” (trade name, manufactured by Hitachi High-Tech Science Corporation). An electric cooling unit “Polyscience” (trade name, manufactured by Hitachi High-Tech Science Corporation) is used to keep each sample and a reference sample at −10° C. for 1 min at a nitrogen gas flow rate of 30 to 50 mL/min. Next, the temperature is raised from −10° C. to 100° C. at a programing rate of 10° C./min. Then, the temperature is decreased from 100° C. to −10° C. under temperature-fall conditions at −10° C./min. Again, the temperature is raised from −10° C. to 100° C. at a programing rate of 10° C./min. In this way, a melt endothermic curve is obtained. At that time, a peak temperature of the melt endothermic curve when the temperature is raised the second time is adopted as a melting point. Note that when there are a plurality of peak temperatures, a peak temperature at the highest melting temperature is defined as a melting point.

In addition, from the viewpoint of the elasticity and easy finger scooping, it is preferable that the ingredient (A) does not exhibit fluidity at 25° C. and is in a semi-solid or paste form.

Note that the semi-solid or paste form indicates that the hardness at 25° C. as measured with a rheometer (manufactured by RHEOTECH, Inc.) (under measurement conditions in which the tip shape of a pressure-sensing shaft is like a disk with a diameter of 5 mm, the penetration speed is 2 cm/min, and the penetration depth is 3 mm) is from 0.1 N to 10 N.

From the viewpoint of giving suitable elasticity, the oily solid cosmetic of the embodiment preferably includes silicone wax with a hardness of 0.5 N or higher as measured under the above conditions. From the viewpoint of elasticity and easy finger scooping, silicone wax with a hardness of preferably from 0.5 to 5.0 N and more preferably from 0.5 to 3.5 N is included.

From the viewpoint of further improving elasticity, finger scooping, and impact resistance, it is preferable that the oily solid cosmetic of the embodiment includes silicone wax with a melting point of 45° C. or lower and a hardness of 0.5 to 5.0 N as measured under the above conditions.

Regarding the ingredient (A), one kind may be used singly or two or more kinds may be used in combination.

The content of ingredient (A) in the oily solid cosmetic of the embodiment may be from 1 to 25 mass %, preferably from 3 to 20 mass %, and more preferably from 5 to 15 mass % with reference to the total amount of the oily solid cosmetic.

(Crosslinked Silicone Elastomer)

As the crosslinked silicone elastomer, a crosslinked organopolysiloxane (or siloxane) elastomer may be used. The elastomer may be either an emulsion or non-emulsion silicone elastomer. Note that the “emulsion” means a crosslinked organopolysiloxane elastomer having a hydrophilic group (e.g., a polyoxyalkylene or polyglycerin moiety such as polyoxyethylene or polyoxypropylene) at a cross-linked part; and the “non-emulsion” means a crosslinked organopolysiloxane without emulsifying capacity and free of an intrinsic hydrophilic group (e.g., a polyoxyalkylene or polyglycerin moiety) at a cross-linked part.

Examples of the crosslinked silicone elastomer include a dimethicone cross polymer, a (dimethicone/vinyldimethicone) cross polymer, a (dimethicone/phenylvinyldimethicone) cross polymer, a (vinyldimethicone/lauryldimethicone) cross polymer, or a (dimethicone/vinyltrimethyl siloxysilicate) cross polymer.

The crosslinked silicone elastomer may be obtained by a crosslinking reaction in which methyl hydrogen polysiloxane and methyl vinyl polysiloxane, for instance, are subjected to addition polymerization to produce siloxane linkage as a skeleton.

In the oily solid cosmetic of the present embodiment, the crosslinked silicone elastomer may be blended as a mixture obtained by dispersing the elastomer in low-viscosity oil with a viscosity at 25° C. of 5 to 50 mPa·s. Note that the viscosity at 25° C. means a value measured with a Brookfield viscometer (at a rotational speed of 12 rpm).

Examples of the low-viscosity oil include dimethicone, diphenyl siloxy phenyl trimethicone, triethylhexanoin, or squalane.

The mixture may be in a swelling material form such as a gel form or a paste form. A commercially available product is applicable to the mixture and examples of the commercially available product include the following.

KSG-15 (a mixture of a (dimethicone/vinyldimethicone) cross polymer and cyclopentasiloxane; elastomer component: about 5 mass %), KSG-16 (a mixture of a (dimethicone/vinyldimethicone) cross polymer and dimethicone at 6 mPa·s; elastomer component: about 25 mass %), KSG-18A (a mixture of a (dimethicone/phenylvinyldimethicone) cross polymer and diphenyl siloxy phenyl trimethicone; elastomer component: about 15 mass %), KSG-210 (a mixture of a (dimethicone/(PEG-10/15))cross polymer and dimethicone; elastomer component: about 25 mass %), KSG-710 (a mixture of a (dimethicone/polyglycerin-3)cross polymer and dimethicone; elastomer component: about 25 mass %), KSG-41 (a mixture of a (vinyldimethicone/lauryldimethicone) cross polymer and liquid paraffin; elastomer component: about 30 mass %), KSG-42 (a mixture of a (vinyldimethicone/lauryldimethicone) cross polymer and light isoparaffin; elastomer component: about 25 mass %), KSG-43 (a mixture of a (vinyldimethicone/lauryldimethicone) cross polymer and glyceryl tri-2-ethylhexanoate; elastomer component: about 30 mass %), KSG-44 (a mixture of a (vinyldimethicone/lauryldimethicone) cross polymer and squalane; elastomer component: about 5 mass %) (the above materials manufactured by Shin-Etsu Chemical Co., Ltd.).

GRANSIL GCM (a mixture of polysilicon-11 and octamethylcyclotetrasiloxane; elastomer component: about 6 mass %), GRANSIL GCM-5 (a mixture of polysilicon-11 and decamethylcyclopentasiloxane; elastomer component: about 6 mass %), GRANSIL IDS (a mixture of polysilicon-11 and isodecane; elastomer component: about 7 mass %), GRANSIL DMG-6 (a mixture of polysilicon-11 and dimethicone at 6 mPa·s; elastomer component: about 18 mass %), GRANSIL DMG-20 (a mixture of polysilicon-11 and dimethicone at 20 mPa·s; elastomer component: about 25 mass %), GRANSIL DMG-50 (a mixture of polysilicon-11 and dimethicone at 50 mPa·s; elastomer component: about 26 mass %), GRANSIL PM (a mixture of polysilicon-11 and phenyl trimethicone; elastomer component: about 20 mass %), GRANSILININ (a mixture of polysilicon-11 and isononyl isononanate; elastomer component: about 15 mass %) (the above materials manufactured by GRANT, Inc.).

VELVESIL 125 (a mixture of an alkyl(C₃₀₋₄₅) cetearyl dimethicone cross polymer and cyclopentasiloxane; elastomer component: about 12.5 mass %), VELVESIL Plus (a mixture of an alkyl(C₃₀₋₄₅) cetearyl dimethicone cross polymer, cyclopentasiloxane, and PEG/PPG-20/23 dimethicone at 5 mPa·s; elastomer component: about 20 mass %), VELVESIL DM (a mixture of a cetearyl dimethicone cross polymer and dimethicone at 5 mPa·s; elastomer component: about 17 mass %) (the above materials manufactured by Momentive Performance Materials, Inc.), BELSIL EG 7 (a mixture of a (dimethicone/vinyldimethicone) cross polymer and dimethicone at 5 mPa·s; elastomer component: about 22 mass %), BELSIL REG 1102 (a mixture of a (dimethicone/vinyltrimethyl siloxysilicate) cross polymer and dimethicone at 5 mPa·s; elastomer component: about 16 mass %) (the above materials manufactured by Wacker Asahikasei Silicone Co., Ltd.),

“Silicone elastomer blend 9045” (a mixture of a dimethicone cross polymer and decamethylcyclopentasiloxane where the crosslinked material has an (actual content) of 12.5%), “silicone elastomer blend 9041” (a mixture of a dimethicone cross polymer and dimethicone at 5 mPa·s where the crosslinked material has an (actual content) of 16%), or a “silicone elastomer blend 9040” (a mixture of a dimethicone cross polymer and decamethylcyclopentasiloxane where the crosslinked material has an (actual content) of 12%) (the above materials manufactured by Dow Corning Toray Silicone Co., Ltd.)

The content (elastomer component) of the ingredient (B) in the oily solid cosmetic of the embodiment may be from 3 to 10 mass % and, from the viewpoint of giving elasticity, is preferably from 4 to 8 mass % and more preferably from 5 to 7 mass % with reference to the total amount of the oily solid cosmetic.

In the oily solid cosmetic of the embodiment, a mixture of low-viscosity oil with a viscosity at 25° C. of 5 to 50 mPa·s and the crosslinked silicone elastomer dispersed in the low-viscosity oil is blended in an amount of preferably from 20 to 45 mass % and more preferably from 25 to 40 mass % with reference to the total amount of the oily solid cosmetic.

In this case, the content of the crosslinked silicone elastomer (elastomer component) in the mixture is preferably from 10 to 30 mass % and more preferably from 15 to 25 mass % with reference to the total amount of the mixture. If the content of the elastomer component is within the above range, it is easy to establish both suitable elasticity and easy scooping.

In addition, the mixture has a hardness at 25° C. of preferably from 0.05 to 0.5 N, more preferably from 0.05 to 0.3 N, and still more preferably from 0.1 to 0.2 N as measured with a rheometer (manufactured by RHEOTECH, Inc,) (under measurement conditions in which the tip shape of a pressure-sensing shaft is like a sphere with a diameter of 10 mm, the penetration speed is 6 cm/min, and the penetration depth is 10 mm). Blending such a mixture in the above blending amount makes it easy to establish both suitable elasticity and easy scooping,

As the mixture that meets the above conditions, the above-mentioned commercially available products may each be used.

The total content of the ingredient (A) and the mixture containing the ingredient (B) in the oily solid cosmetic of the embodiment may be from 30 mass % to 55 mass % and, from the viewpoint of giving elasticity, more preferably from 35 mass % to 45 mass % with reference to the total amount of the oily solid cosmetic.

Examples of a suitable combination of the ingredient (A) and the mixture containing the ingredient (B) include the following (i) to (iii):

(i) a combination of 100 parts by mass of (A) silicone wax with a hardness of 0.5 to 5.0 N and 200 to 500 parts by mass of a mixture with a hardness of 0.05 to 0.5 N;

(ii) a combination of 100 parts by mass of (A) silicone wax with a hardness of more than 5.0 N and 500 to 2000 parts by mass of a mixture with a hardness of 0.05 to 0.3 N; and

(iii) a combination of 100 parts by mass of (A) silicone wax with a hardness of more than 5.0 N and 200 to 500 parts by mass of a mixture with a hardness of 0.05 to 0.1 N.

Note that the hardness of the above ingredient (A) and the hardness of the mixture each mean a hardness at 25° C. as measured under the above-mentioned conditions.

(Powder Ingredient)

The content of the powder ingredient may be from 40 to 70 mass % and, from the viewpoint of formability, preferably from 50 to 60 mass % with reference to the total amount of the oily solid cosmetic. In addition, the powder ingredient in the oily solid cosmetic of the present embodiment contains sparkling powder, and the content of the sparkling powder may be 30 mass % or more and, from the viewpoint of establishing both given pearl feeling and formability, preferably from 5 to 50 mass % and more preferably from 30 to 50 mass % with reference to the total amount of the oily solid cosmetic.

Examples of the sparkling powder include bismuth oxychloride, mica titanium, titanium dioxide-coated bismuth oxychloride, iron oxide-coated mica, iron oxide-coated mica titanium, red iron oxide-coated mica titanium, red iron oxide/black iron oxide-coated mica titanium, organic pigment-coated mica titanium, fish scale guanine, iron oxide-coated synthetic phlogopite, aluminum flake, titanium dioxide-coated glass flake, polyethylene terephthalate/aluminum/epoxy laminated powder, or polyethylene terephthalate/polyolefin laminated film powder.

It is preferable that the sparkling powder is flake-shaped sparkling powder with an average particle size of 10 to 150 μm. In addition, the aspect ratio (length/thickness) is preferably from 5 to 1000 and more preferably from 10 to 300 in view of uniform scooping and tight adhesion.

The powder ingredient may contain powder other than the sparkling powder. Such powder may be used without particular limitation if the powder is usually used for cosmetics. Examples include body powder, white pigment, or color pigment. The shape of the powder is not particularly limited, and the powder may have, for instance, a spherical, plate-like, or needle-like shape, an atomized, microparticle, or pigment-grade particle size, and/or a porous or nonporous particle structure.

Specific examples include: body pigments such as mica, synthetic mica, sericite, kaoline, silicon carbide, barium sulfate, bentonite, smectite, aluminum oxide, silica, magnesium oxide, zirconium oxide, magnesium carbonate, calcium carbonate, chromium oxide, aluminum magnesium hydroxide; ultraviolet scattering agents such as microparticle titanium oxide, microparticle zinc oxide; organic powders such as nylon powder, polymethyl methacrylate powder, acrylonitrile-methacrylic acid copolymer powder, a vinylidene chloride-methacrylic acid copolymer, polyethylene powder, polystyrene powder, organopolysiloxane elastomer powder, polymethylsilsesquloxane powder, urethane powder, wool powder, silk powder, cellulose powder, N-acyllysine powder; complex powders such as microparticle titanium oxide-coated mica titanium, microparticle titanium oxide-coated nylon, barium sulfate-coated mica titanium, titanium oxide-containing silica, zinc oxide-containing silica; or metal soaps such as magnesium stearate, zinc myristate, aluminum stearate, and calcium stearate.

Examples of the color pigment include: inorganic color pigments such as red iron oxide, yellow iron oxide, black iron oxide, cobalt oxide, chromium oxide, ultramarine, iron blue, titanium oxide; organic color pigments such as Red No. 228, Red No. 226, Blue No. 404, Red No. 201, Red No. 202, Yellow No. 4 aluminum lake; or natural dyes such as carmine and safflower.

Regarding the powder ingredient, one kind may be used singly or two or more kinds may be used in combination.

(Additional Oily Ingredient)

In the oily solid cosmetic of the present embodiment, an oily ingredient such as solid oil or an oil solution other than the solid oil in addition to the above ingredient (A) may be used without particular limitation if the oil solution is usually used for cosmetics. Regarding the oily ingredient, one kind may be used singly or two or more kinds may be used in combination.

Examples of the solid oil include: hydrocarbons such as paraffin wax, microcrystalline wax, polyethylene; plant-derived fats such as hydrogenated castor oil, hydrogenated jojoba oil, carnauba wax, rice wax; esters such as glyceryl tribehenate, cholesterol fatty acid ester; higher fatty acids such as stearic acid, behenic acid; higher alcohols such as stearyl alcohol, behenyl alcohol; silicones such as alkyl-modified silicone other than the above ingredient (A), acryl-modified silicone other than the above ingredient (A); or sugar fatty acid esters such as dextrin palmitate and inulin stearate. Regarding the above, one kind may be used singly or two or more kinds may be used in combination.

As the oil solution other than the solid oil, paste oil or liquid oil may be used. Examples of the paste oil include vaseline, hexa(hydroxystearic acid/stearic acid/rosin acid) dipentaerythrityl, tetra(hydroxystearic acid/isostearic acid) dipentaerythrityl, dipentaerythrityl pentaisostearate, dipentaerythrityl hexahydroxystearate, tri(caprylic acid/capric acid/myristic acid/stearic acid) glyceryl, isostearic acid hydrogenated castor oil, phytosteryl oleate, hexa(oleic acid/palmitic acid/stearic acid) sucrose, dimerdilinoleyl dimerdilinolate bis(behenyl/isostearyl/phytosteryl), dimerdilinoleic acid (phytosteryl/isostearyl/cetyl/stearyl/behenyl), dimerdilinoleic acid hydrogenated castor oil, macadamia nut fatty acid phytosteryl, or bis-diglyceryl polyacyl adipate-2.

Examples of the liquid oil include: ester oils such as cetyl ethylhexanoate, ethylhexyl palmitate, triethylhexanoin, isotridecyl isononanate, isostearyl isostearate, neopentylglycol diethylhexanoate, tri(caprylic/capric acid) glyceryl, neopentylglycol dicaprylate, propanediol diisostearate, trimethylolpropane triethylhexanoate, octyldodecyl myristate, octyldodecyl stearoyloxy stearate, diisostearyl malate, polyglyceryl triisostearate, dipentaerythrityl pentaisostearate, trimethylolpropane triisostearate; silicone oils such as dimethicone, methylphenyl polysiloxane; hydrocarbon oils such as liquid paraffin, squalane, an olefin oligomer, hydrogenated polyisobutene, hydrogenated polydecene; plant oils such as sunflower seed oil, jojoba seed oil, olive oil, castor oil; higher fatty acids such as isostearic acid; or higher alcohols such as isostearyl alcohol, octyldodecanol, and oleyl alcohol.

The content of the oily ingredient, which is other than the above ingredient (A), in the oily solid cosmetic of the present embodiment is preferably from 20 to 55 mass % and more preferably from 25 to 50 mass % with reference to the total amount of the oily solid cosmetic.

From the viewpoint of workability during powder ingredient dispersion, the oily solid cosmetic of the present embodiment may contain a surfactant.

Examples of the surfactant that can be used include a hydrophilic nonionic surfactant, an anionic surfactant, a cationic surfactant, or an amphoteric surfactant. Examples of the hydrophilic nonionic surfactant include polyoxyalkylene alkyl ether, glycerin alkyl ether, glycerin fatty acid ester, polyglycerin fatty acid ester, sorbitan fatty acid ester, and alkylene glycol adducts thereof, polyalkylene glycol fatty acid ester, polyglycerin-modified silicone, or polyether-modified silicone. Examples of the anionic surfactant include alkyl phosphate, polyoxyalkylene alkyl ether phosphate, sulfonate, alkyl sulfate, or polyaspartate. Examples of the cationic surfactant include an alkylamine salt or an alkyltrimethylammonium salt. Examples of the amphoteric surfactant include lecithin, a carbobetaine-type amphoteric surfactant, a sulfobetaine-type amphoteric surfactant, or an amino acid-type amphoteric surfactant. Regarding the surfactant, one kind may be used singly or two or more kinds may be used in combination.

Among the above surfactants, polyether-modified silicone is preferable from the viewpoint of powder ingredient dispersion property. Examples of the polyether-modified silicone include PEG-10 dimethicone, PEG-9 dimethicone, PEG-3 dimethicone, PEG-11, methyl ether dimethicone, PEG-9 polydimethylsiloxyethyl dimethicone, or lauryl PEG-9 polydimethylsiloxyethyl dimethicone.

When the oily solid cosmetic of the present embodiment contains a surfactant, the content may be 2.0 mass % or less and, from the viewpoint of water resistance, preferably 0.5 mass % or less with reference to the total amount of the oily solid cosmetic.

The oily solid cosmetic of the present embodiment may contain, in addition to the above ingredients, components usually used for cosmetics, such as a preservative, an antioxidant, a dye, a thickener, a pH modifier, a perfume, a UV absorber, a moisturizing agent, a chelator, and/or an antiphlogistic.

The oily solid cosmetic of the present embodiment is fit for, for instance, foundation, cheek, face color, eye shadow, eyebrow, lipstick, and/or concealer makeup cosmetics.

Next, a process for producing an oily solid cosmetic according to an embodiment of the invention will be described.

The process for producing an oily solid cosmetic according to the present embodiment includes a step of causing a cosmetic base to be compacted, The cosmetic base may contain: the above-described ingredient (A); the ingredient (B) or a mixture of the ingredient (B) and low-viscosity oil; the powder ingredient; and optionally an additional ingredient.

The composition of the cosmetic base may be set, if appropriate, so as to obtain the above-described oily solid cosmetic of the present embodiment. The blending amount of each ingredient in the cosmetic base may be likewise set to be within the above-described preferable ranges in the oily solid cosmetic.

For instance, the content of the powder ingredient in the cosmetic base may be from 40 to 70 mass % with reference to the total amount of the cosmetic base. In addition, the content of the sparkling powder in the cosmetic base may be 30 mass % or higher with reference to the total amount of the cosmetic base. Further, the content of the ingredient (A) in the cosmetic base may be from 1 to 25 mass % with reference to the total amount of the cosmetic base. Furthermore, the content of the ingredient (B) in the cosmetic base may be from 3 to 10 mass % with reference to the total amount of the cosmetic base. Moreover, the ingredient (B) may be blended, as a mixture dispersed in low-viscosity oil, in the cosmetic base. In this case, the content of the mixture in the cosmetic base may be from 20 to 45 mass % with reference to the total amount of the cosmetic base.

The cosmetic base may be a slurry. The slurry may be prepared by a process including mixing: the above-described ingredient (A); the ingredient (B) or a mixture of the ingredient (B) and low-viscosity oil; the powder ingredient; and optionally an additional ingredient. The mixing may be carried out by undergoing: a step of mixing the powder ingredient to obtain a first mixture; a step of mixing the ingredient (A), the ingredient (B) or a mixture of the ingredient (B) and low-viscosity oil, and an optionally blended additional ingredient to obtain a second mixture; and a step of blending the first mixture and the second mixture.

The step of obtaining the first mixture may be performed using, for instance, a super mixer or a Henschel mixer, and an atomizer, if necessary, may be used for pulverization.

The step of obtaining the second mixture may be performed using, for instance, a disperser or a homomixer, and the above respective ingredients may be mixed at from 25 to 50° C.

The step of blending the first mixture and the second mixture may be performed using, for instance, a super mixer and/or a Henschel mixer, and an atomizer, if necessary, may be used for pulverization.

The cosmetic base slurry may contain a dispersion medium to be removed after compacted. When liquid oil is blended as an oily ingredient, the slurry may be prepared without using such a dispersion medium.

The cosmetic base may be compacted by defoaming, if necessary, the cosmetic base slurry, loading the resulting slurry into a given container, and pressing the slurry by vacuum compaction, etc. The pressing conditions are not particularly limited and are, for instance, conditions at room temperature and at a pressure of 2 to 6 kgf/cm². The pressing may be conducted once or twice or more. The compacted cosmetic base may be dried.

Examples of the given container include a medium-sized dish such as a metal dish or a resin dish.

### EXAMPLES

Hereinafter, the present invention will be further described in detail by referring to Examples. However, the technical scope of the invention is not limited by these Examples. Note that the numbers in Tables each indicate a content (mass %) with reference to the total amount of the cosmetic base.

Before explanation of the Examples, evaluation methods used in each Example are described.

(1) Formability

Each cosmetic base slurry was filled into a metal dish, and the surface uniformity and the presence or absence of crack, peeling, and/or fissure after compaction were visually inspected. Each sample was scored and rated by four grades according to the following evaluation standards, and the average (n=5) was determined in accordance with the following criteria.

### [Evaluation Standards]

4 Points: No change, uniform.

3 Points: Occurrence of crack, peeling, and/or fissure, etc., is slightly observed.

2 Points: Occurrence of crack, peeling, and/or fissure, etc., is observed.

1 Point. Incidence of crack, peeling, and/or fissure, etc., is very frequent.

### [Criteria (Score Average)]

⊙: 3.5 or higher.

∘: From 3.0 to less than 3.5.

Δ: From 2.0 to less than 3.0.

×: Less than 2.0.

(2) Use Performance and Use Feeling (Elasticity, Finger Scooping)

Twenty cosmetic evaluation professional panelists were each instructed to use each oily solid cosmetic sample by a finger and to conduct 5-grade evaluation and score each sample according to the following evaluation standards with respect to the elasticity (whether elasticity can be felt when a finger is used to touch the cosmetic) and the finger scooping (whether an appropriate amount can be scooped when a finger is used to scoop the cosmetic). Further, the scores of all the panelists were averaged and determined in accordance with the following criteria.

### [Evaluation Standards]

5 Points: Excellent.

4 Points: Good.

3 Points: Acceptable.

2 Points: Poor.

1 Point: Bad.

### [Criteria (Score Average)]

⊙: 4 or higher.

∘: From 3 to less than 4.

Δ: From 2 to less than 3.

×: Less than 2.

(3) Use Performance and Use Feeling (Spreadability, Tight Adhesion, Moist Feeling, Less Pearl Irregularity)

Twenty cosmetic evaluation professional panelists were each instructed to use each oily solid cosmetic sample by a finger and to conduct 5-grade evaluation and score each sample according to the following evaluation standards with respect to the spreadability, tight adhesion, moist feeling, and less pearl irregularity. Further, the scores of all the panelists were averaged and determined in accordance with the following criteria.

### [Evaluation Standards]

5 Points: Excellent.

4 Points: Good.

3 Points: Acceptable.

2 Points: Poor.

1 Point: Bad.

### [Criteria (Score Average)]

⊙: 4 or higher.

∘: From 3 to less than 4.

Δ: From 2 to less than 3.

×: Less than 2.

(4) Impact Resistance (Drop Resistance)

Each oily solid cosmetic sample was made to fall 10 times on a P tile from the height of 70 cm with the content material faced upward. Then, the resulting state was inspected, and 4-grade evaluation was conducted according to the following evaluation standards. Each sample was scored and the average (n=5) was determined in accordance with the following criteria.

### [Evaluation Standards]

4 Points: No change.

3 Points: Somewhat detached, chipped, and/or cracked but there is no problem.

2 Points: Partially detached, chipped, and/or cracked.

1 Point: Totally detached, chipped, and/or cracked.

### [Criteria (Score Average)]

⊙: 3.5 or higher.

∘: From 3.0 to less than 3.5.

Δ: From 2.0 to less than 3.0.

×: Less than 2.0.

### Examples 1 to 19 and Comparative Examples 1 to 6

Eye shadows with compositions listed in Tables 1 to 6 were prepared by the following production process and were evaluated as above. Tables 1 to 6 collectively show the results.

<Production Process>

The ingredients 1 to 15 were homogenously dispersed at 50° C. to produce each dispersion I. The powder ingredients of the ingredients 16 to 22 were homogenously dispersed with a Henschel mixer to produce each mixture II. The dispersion I and the mixture II were blended to prepare a cosmetic base slurry. The cosmetic base slurry was filled into a metal dish and was compacted under the following pressing conditions to produce each oily solid cosmetic.

Pressing conditions: pressed twice for 2 sec at a load of 3 kgf/cm².

TABLE 1

| | | | Example | | | | | |
|---|---|---|---|---|---|---|---|---|
| | | | 1 | 2 | 3 | 4 | 5 | 6 |
| 1 | (A) | Silicone wax A1 | 10 | 10 | 10 | 10 | — | — |
| 2 | | Silicone wax A2 | — | — | — | — | — | 10 |
| 3 | | Silicone wax A3 | — | — | — | — | — | — |
| 4 | | Silicone wax A4 | — | — | — | — | — | — |
| 5 | | Silicone wax A5 | — | — | — | — | 4 | — |
| 6 | Hexa(hydroxystearic acid/stearic | | — | — | — | — | — | — |
| | acid/rosin acid)dipentaerythrityl | | | | | | | |
| 7 | Tri(caprylic acid/capric acid/myristic | | — | — | — | — | — | — |
| | acid/stearic acid)glyceryl | | | | | | | |
| 8 | Lauric acid | | — | — | — | — | — | — |
| 9 | (B) | Silicone elastomer B1 | 25 | 30 | 35 | 40 | 40 | 4 |
| 10 | | Silicone elastomer B2 | — | — | — | — | — | — |
| 11 | | Silicone elastomer B3 | — | — | — | — | — | — |
| 12 | | Silicone elastomer B4 | — | — | — | — | — | — |
| 13 | | Silicone elastomer B5 | — | — | — | — | — | — |
| 14 | Diisostearyl malate | | 10 | 5 | 1 | 1 | 1 | 1 |
| 15 | PEG-10 dimethicone | | 1 | 1 | 1 | 1 | 1 | 1 |
| 16 | Powder | Synthetic phlogopite | 6.5 | 6.5 | 5.5 | 6.5 | 6.5 | 5.5 |
| 17 | ingredient | Methyl polymethacryla | 2 | 2 | 2 | 2 | 2 | 2 |
| 18 | | Red iron oxide | 2.5 | 2.5 | 2.5 | 2.5 | 2.5 | 2.5 |
| 19 | | Yellow iron oxide | 2 | 2 | 2 | 2 | 2 | 2 |
| 20 | | Black iron oxide | 1 | 1 | 1 | 1 | 1 | 1 |
| 21 | | Red iron oxide-coated | 20 | 20 | 20 | 17 | 20 | 15 |
| | | mica titanium | | | | | | |
| 22 | | Red iron oxide/black | 20 | 20 | 20 | 17 | 20 | 15 |
| | | iron oxide-coated | | | | | | |
| | | Mica titanium | | | | | | |
| Evaluation | (1) | Formability | ⊙ | ⊙ | ⊙ | ⊙ | ◯ | ⊙ |
| | (2) | Elasticity | ⊙ | ⊙ | ⊙ | ◯ | ◯ | ◯ |
| | | Uniform finger | ⊙ | ⊙ | ⊙ | ◯ | ◯ | ⊙ |
| | | scooping | | | | | | |
| | (3) | Spreadability | ⊙ | ⊙ | ⊙ | ⊙ | ⊙ | ⊙ |
| | | Tight adhesion | ⊙ | ⊙ | ⊙ | ⊙ | ⊙ | ◯ |
| | | Less pearl irregularity | ⊙ | ⊙ | ⊙ | ⊙ | ⊙ | ⊙ |
| | | Moist finish | ⊙ | ⊙ | ⊙ | ⊙ | ◯ | ◯ |
| | (4) | Impact resistance | ⊙ | ⊙ | ⊙ | ⊙ | ◯ | ⊙ |

TABLE 2

| | | | Example | | | | | |
|---|---|---|---|---|---|---|---|---|
| | | | 7 | 8 | 9 | 10 | 11 | 12 |
| 1 | (A) | Silicone wax A1 | — | 10 | 22 | 10 | 17 | 10 |
| 2 | | Silicone wax A2 | 10 | — | — | — | — | — |
| 3 | | Silicone wax A3 | — | — | — | — | — | — |
| 4 | | Silicone wax A4 | — | — | — | — | — | — |
| 5 | | Silicone wax A5 | — | — | — | — | — | — |
| 6 | Hexa(hydroxystearic acid/stearic | | — | — | — | — | — | — |
| | acid/rosin acid)dipentaerythrityl | | | | | | | |
| 7 | Tri(caprylic acid/capric acid/myristic | | — | — | — | — | — | — |
| | acid/stearic acid)glyceryl | | | | | | | |
| 8 | Lauric acid | | — | — | — | — | — | — |
| 9 | (B) | Silicone elastomer B1 | 35 | 20 | 22 | — | — | — |
| 10 | | Silicone elastomer B2 | — | — | — | 20 | — | — |
| 11 | | Silicone elastomer B3 | — | — | — | — | 27 | — |
| 12 | | Silicone elastomer B4 | — | — | — | — | — | 20 |
| 13 | | Silicone elastomer B5 | — | 15 | — | 15 | — | 15 |
| 14 | Diisostearyl malate | | 1 | 1 | 1 | 1 | 1 | 1 |
| 15 | PEG-10 dimethicone | | 1 | 1 | 1 | 1 | 1 | 1 |
| 16 | Powder | Synthetic phlogopite | 5.5 | 5.5 | 6.5 | 5.5 | 6.5 | 5.5 |
| 17 | ingredient | Methyl polymethacryla | 2 | 2 | 2 | 2 | 2 | 2 |
| 18 | | Red iron oxide | 2.5 | 2.5 | 2.5 | 2.5 | 2.5 | 2.5 |
| 19 | | Yellow iron oxide | 2 | 2 | 2 | 2 | 2 | 2 |
| 20 | | Black iron oxide | 1 | 1 | 1 | 1 | 1 | 1 |
| 21 | | Red iron oxide-coated | 20 | 20 | 20 | 20 | 20 | 20 |
| | | mica titanium | | | | | | |
| 22 | | Red iron oxide/black | 20 | 20 | 20 | 20 | 20 | 20 |
| | | iron oxide-coated | | | | | | |
| | | Mica titanium | | | | | | |
| Evaluation | (1) | Formability | ⊙ | ⊙ | ⊙ | ⊙ | ⊙ | ⊙ |
| | (2) | Elasticity | ◯ | ⊙ | ◯ | ⊙ | ⊙ | ◯ |
| | | Uniform finger | ◯ | ⊙ | ⊙ | ⊙ | ◯ | ⊙ |
| | | scooping | | | | | | |
| | (3) | Spreadability | ⊙ | ⊙ | ⊙ | ⊙ | ⊙ | ⊙ |
| | | Tight adhesion | ◯ | ⊙ | ⊙ | ⊙ | ⊙ | ⊙ |
| | | Less pearl irregularity | ⊙ | ⊙ | ⊙ | ⊙ | ⊙ | ⊙ |
| | | Moist finish | ◯ | ⊙ | ⊙ | ⊙ | ⊙ | ⊙ |
| | (4) | Impact resistance | ⊙ | ⊙ | ⊙ | ⊙ | ⊙ | ⊙ |

TABLE 3

| | | | Example | | | | |
|---|---|---|---|---|---|---|---|
| | | | 13 | 14 | 15 | 16 | 17 |
| 1 | (A) | Silicone wax A1 | 1.5 | 5 | 17 | — | — |
| 2 | | Silicone wax A2 | — | — | — | 5 | 10 |
| 3 | | Silicone wax A3 | — | — | — | — | — |
| 4 | | Silicone wax A4 | — | — | — | — | — |
| 5 | | Silicone wax A5 | — | — | — | — | — |
| 6 | Hexa(hydroxystearic acid/stearic | | 8.5 | — | — | — | — |
| | acid/rosin acid)dipentaerythrityl | | | | | | |
| 7 | Tri(caprylic acid/capric acid/myristic | | — | — | — | — | — |
| | acid/stearic acid)glyceryl | | | | | | |
| 8 | Lauric acid | | — | — | — | — | — |
| 9 | (B) | Silicone elastomer B1 | 35 | 35 | 27 | 20 | — |
| 10 | | Silicone elastomer B2 | — | — | — | — | — |
| 11 | | Silicone elastomer B3 | — | — | — | — | — |
| 12 | | Silicone elastomer B4 | — | — | — | — | 20 |
| 13 | | Silicone elastomer B5 | — | — | — | 15 | 15 |
| 14 | Diisostearyl malate | | 1 | 6 | 1 | 6 | 1 |
| 15 | PEG-10 dimethicone | | 1 | 1 | 1 | 1 | 1 |
| 16 | Powder | Synthetic phlogopite | 5.5 | 5.5 | 6.5 | 5.5 | 5.5 |
| 17 | ingredient | Methyl polymethacryla | 2 | 2 | 2 | 2 | 2 |
| 18 | | Red iron oxide | 2.5 | 2.5 | 2.5 | 2.5 | 2.5 |
| 19 | | Yellow iron oxide | 2 | 2 | 2 | 2 | 2 |
| 20 | | Black iron oxide | 1 | 1 | 1 | 1 | 1 |
| 21 | | Red iron oxide-coated | 20 | 20 | 20 | 20 | 20 |
| | | mica titanium | | | | | |
| 22 | | Red iron oxide/black | 20 | 20 | 20 | 20 | 20 |
| | | iron oxide-coated | | | | | |
| | | Mica titanium | | | | | |
| Evaluation | (1) | Formability | ⊙ | ⊙ | ⊙ | ◯ | ⊙ |
| | (2) | Elasticity | ⊙ | ⊙ | ◯ | ⊙ | ◯ |
| | | Uniform finger | ◯ | ⊙ | ⊙ | ⊙ | ⊙ |
| | | scooping | | | | | |
| | (3) | Spreadability | ⊙ | ⊙ | ⊙ | ⊙ | ⊙ |
| | | Tight adhesion | ⊙ | ⊙ | ⊙ | ⊙ | ⊙ |
| | | Less pearl irregularity | ◯ | ⊙ | ⊙ | ⊙ | ⊙ |
| | | Moist finish | ⊙ | ⊙ | ⊙ | ⊙ | ⊙ |
| | (4) | Impact resistance | ◯ | ◯ | ⊙ | ◯ | ⊙ |

TABLE 4

| | | | Example | |
|---|---|---|---|---|
| | | | 18 | 19 |
| 1 | (A) | Silicone wax Al | — | — |
| 2 | | Silicone wax A2 | — | — |
| 3 | | Silicone wax A3 | 10 | — |
| 4 | | Silicone wax A4 | — | 10 |
| 5 | | Silicone wax A5 | — | — |
| 6 | Hexa(hydroxystearic acid/stearic | | — | — |
| | acid/rosin acid)dipentaerythrityl | | | |
| 7 | Tri(caprylic acid/capnc | | — | — |
| | acid/myristic acid/stearic | | | |
| | acid)glyceryl | | | |
| 8 | Lauric acid | | — | — |
| 9 | (B) | Silicone elastomer B1 | 35 | 35 |
| 10 | | Silicone elastomer B2 | — | — |
| 11 | | Silicone elastomer B3 | — | — |
| 12 | | Silicone elastomer B4 | — | — |
| 13 | | Silicone elastomer B5 | — | — |
| 14 | Diisostearyl malate | | 1 | 1 |
| 15 | PEG-10 dimethicone | | 1 | 1 |
| 16 | Powder | Synthetic phlogopite | 5.5 | 5.5 |
| 17 | ingredient | Methyl | 2 | 2 |
| | | polymethacrylate | | |
| 18 | | Red iron oxide | 2.5 | 2.5 |
| 19 | | Yellow iron oxide | 2 | 2 |
| 20 | | Black iron oxide | 1 | 1 |
| 21 | | Red iron oxide-coated | 20 | 20 |
| | | mica titanium | | |
| 22 | | Red iron oxide/black | 20 | 20 |
| | | iron oxide-coated | | |
| | | mica titanium | | |
| Evaluation | (l) | Formability | ⊙ | ⊙ |
| | (2) | Elasticity | ○ | ⊙ |
| | | Uniform finger scooping | ○ | ⊙ |
| | (3) | Spreadability | ⊙ | ⊙ |
| | | Tight adhesion | ⊙ | ⊙ |
| | | Less pearl irregularity | ⊙ | ⊙ |
| | | Moist finish | ⊙ | ⊙ |
| | (4) | Impact resistance | ○ | ⊙ |

TABLE 5

| | | | Comparative Example | | | | | |
|---|---|---|---|---|---|---|---|---|
| | | | 1 | 2 | 3 | 4 | 5 | 6 |
| 1 | (A) | Silicone wax A1 | 45 | — | — | — | — | — |
| 2 | | Silicone wax A2 | — | — | — | — | — | — |
| 3 | | Silicone wax A3 | — | — | — | — | — | — |
| 4 | | Silicone wax A4 | — | — | — | — | — | — |
| 5 | | Silicone wax A5 | — | — | — | — | — | — |
| 6 | Hexa(hydroxystearic acid/stearic | | — | 10 | — | — | — | — |
| | acid/rosin acid)dipentaerythrityl | | | | | | | |
| 7 | Tri(caprylic acid/capric acid/myristic | | — | — | 10 | — | — | — |
| | acid/stearic acid)glyceryl | | | | | | | |
| 8 | Lauric acid | | — | — | — | 10 | 10 | — |
| 9 | (B) | Silicone elastomer B1 | — | 35 | 35 | 35 | 45 | 45 |
| 10 | | Silicone elastomer B2 | — | — | — | — | — | — |
| 11 | | Silicone elastomer B3 | — | — | — | — | — | — |
| 12 | | Silicone elastomer B4 | — | — | — | — | — | — |
| 13 | | Silicone elastomer B5 | — | — | — | — | — | — |
| 14 | Diisostearyl malate | | 1 | 1 | 1 | 1 | 1 | 1 |
| 15 | PEG-10 dimethicone | | 1 | 1 | 1 | 1 | 1 | 1 |
| 16 | Powder | Synthetic phlogopite | 5.5 | 5.5 | 5.5 | 5.5 | 5.5 | 5.5 |
| 17 | ingredient | Methyl polymethacryla | 2 | 2 | 2 | 2 | 2 | 2 |
| 18 | | Red iron oxide | 2.5 | 2.5 | 2.5 | 2.5 | 2.5 | 2.5 |
| 19 | | Yellow iron oxide | 2 | 2 | 2 | 2 | 2 | 2 |
| 20 | | Black iron oxide | 1 | 1 | 1 | 1 | 1 | 1 |
| 21 | | Red iron oxide-coated | 20 | 20 | 20 | 20 | 15 | 20 |
| | | mica titanium | | | | | | |
| 22 | | Red iron oxide/black iron | 20 | 20 | 20 | 20 | 15 | 20 |
| | | oxide-coated | | | | | | |
| | | Mica titanium | | | | | | |
| Evaluation | (1) | Formability | ◯ | ⊙ | Δ | x | x | ⊙ |
| | (2) | Elasticity | x | ⊙ | Δ | x | Δ | Δ |
| | | Uniform finger scooping | Δ | x | Δ | x | Δ | Δ |
| | (3) | Spreadability | x | Δ | Δ | x | Δ | ◯ |
| | | Tight adhesion | ⊙ | ◯ | Δ | x | Δ | x |
| | | Less pearl irregularity | ◯ | x | Δ | Δ | Δ | Δ |
| | | Moist finish | ⊙ | ◯ | x | x | x | Δ |
| | (4) | Impact resistance | ◯ | ⊙ | ◯ | Δ | Δ | ◯ |

In Tables 1 to 5, the specifics of each ingredient are as follows.

Silicone wax A1: BELSIL CDM 3526 VP (trade name, alkyl(C₂₆₋₂₈) dimethicone, manufactured by Dow Corning Toray Co., Ltd.) with a melting point of 43.4° C. and a hardness of 0.6273N and in a paste form at 25° C.

Silicone wax A2: BELSIL SDM 5055 VP (trade name, stearyl dimethicone, manufactured by Dow Corning Toray Co., Ltd.) with a melting point of 30.7° C. and a hardness of more than 19.6 N and in a solid to semi-solid form at 25° C.

Silicone wax A3: KF-7002 (trade name, a (stearoxy methicone/dimethicone) copolymer, manufactured by Shin-Etsu Chemical Co., Ltd.) with a melting point of 54.5° C. and a hardness of 3.626 N and in a semi-solid form at 25° C.

Silicone wax A4: KP-561P (trade name, a (acrylate/stearyl acrylate/dimethicone methacrylate) copolymer, manufactured by Shin-Etsu Chemical Co., Ltd.) with a melting point of 29.7° C. and a hardness of 3.332 N and in a semi-solid form at 25° C.

Silicone wax A5: KP-562P (trade name, a (acrylate/behenyl acrylate/dimethicone methacrylate) copolymer, manufactured by Shin-Etsu Chemical Co., Ltd.) with a melting point of 50.4° C. and a hardness of more than 19.6 N and in a solid form at 25° C.

Hexa(hydroxystearic acid/stearic acid/rosin acid) dipentaerythrityl: COSMOL 168ARV (trade name, manufactured by the Nisshin OilliO Group, Ltd.) with a melting point of 25.9° C. and a hardness of 0.9996 N and in a paste form at 25° C.

Tri(cap ylic acid/capric acid/myristic acid/stearic acid)glyceryl: SARACOS 334 (trade name, manufactured by the Nisshin OilliO Group, Ltd.) with a melting point of 37.3° C. and a hardness of 8.624 N and in a semi-solid form at 25° C.

Lauric acid: NAA-122 (trade name, manufactured by NOF CORPORATION) with a melting point of 47.8° C. and a hardness of more than 19.6 N and in a solid form at 25° C.

The melting point and the hardness of each oily ingredient were measured by the following protocols.

(To Measure Melting Point)

About 5 mg of each sample was weighed and then put into an aluminum sample pan. An aluminum cover is attached to this pan, which is then installed in a differential scanning calorimeter “DSC7020” (trade name, manufactured by Hitachi High-Tech Science Corporation). An electric cooling unit “Polyscience” (trade name, manufactured by Hitachi High-Tech Science Corporation) was used to keep each sample and a reference sample at −10° C. for 1 min at a nitrogen gas flow rate of 30 to 50 mL/min. Next, the temperature was raised from −10° C. to 100° C. at a programing rate of 10° C./min. Then, the temperature was decreased from 100° C. to -10° C. under temperature-fall conditions at −10° C./min. Again, the temperature was raised from −10° C. to 100° C. at a programing rate of 10° C./min. In this way, a melt endothermic curve was obtained. At that time, a peak temperature of the melt endothermic curve when the temperature was raised the second time was adopted as a melting point. Note that when there were a plurality of peak temperatures, a peak temperature at the highest melting temperature was defined as a melting point.

(To Measure Hardness)

A rheometer (manufactured by RHEOTECH, Inc.) was used to measure a hardness under conditions in which the tip shape of a pressure-sensing shaft was like a disk with a diameter of 5 mm, the penetration speed was 2 cm/min, the penetration depth was 3 mm, and the temperature was 25° C.

Silicone elastomer B1: 9041 Silicone Elastomer Blend (trade name, a mixture of 16 mass % of dimethicone cross polymer and 84 mass % of dimethicone, manufactured by Dow Corning Toray Co., Ltd.) with a hardness of 0.196 N.

Silicone elastomer B2: BELSIL EG 7 (trade name, a mixture of about 22 mass % of (dimethicone/vinyldimethicone) cross polymer and about 78 mass % of dimethicone, manufactured by Wacker Asahikasei Silicone Co., Ltd.) with a hardness of 0.294 N.

Silicone elastomer B3: BELSIL REG 1102 (trade name, a mixture of about 16 mass % of (dimethicone/vinyltrimethyl siloxysilicate) cross polymer and about 84 mass % of dimethicone, manufactured by Wacker Asahikasei Silicone Co., Ltd.) with a hardness of 0.0784 N.

Silicone elastomer B4: KSG-16 (trade name, a mixture of about 15 mass % of (dimethicone/vinyldimethicone) cross polymer and about 85 mass % of dimethicone, manufactured by Shin-Etsu Chemical Co., Ltd.) with a hardness of 0.0882 N.

Silicone elastomer B5: KSG-18A (trade name, a mixture of about 15 mass % of (dimethicone/phenylvinyldimethicone) cross polymer and about 85 mass % of diphenyl siloxy phenyl trimethicone, manufactured by Shin-Etsu Chemical Co., Ltd.) with a hardness of 0.196 N.

The hardness of each mixture was a value measured using a rheometer (manufactured by RHEOTECH, Inc.) under conditions in which the shape of a pressure-sensing shaft was like a sphere with a diameter of 10 mm, the penetration speed was 6 cm/min, the penetration depth was 10 mm, and the temperature was 25° C.

Tables 1 to 4 have demonstrated that the oily solid cosmetics obtained in Examples 1 to 19 are found to yield sufficient quality in all of (1) formability, (2) elasticity, uniform finger scooping, (3) spreadability, tight adhesion, less pearl irregularity, moist finish, and (4) impact resistance.

(Example 20: Cheek Cosmetic)

| | (Blending ratio |
|---|---|
| (Ingredient) | (mass %)) |
| 1. Silicone wax A1 | 10.00 |
| 2. Silicone elastomer B1 | 30.00 |
| 3. Silicone elastomer B4 | 5.00 |
| 4. Tri(caprylic/capric acid)glyceryl | 5.00 |
| 5. Talc | 10.00 |
| 6. Mica | 10.00 |
| 7. Synthetic phlogopite | 20.50 |
| 8. Silica | 3.00 |
| 9. Mica titanium | 5.00 |
| 10. Red No. 201 | 0.50 |
| 11. Red No. 202 | 0.50 |
| 12. Yellow No. 4 aluminum lake | 0.50 |

<Production Process>

The ingredients 1 to 4 were homogenously dispersed at 50° C. to produce a dispersion I. The powder ingredients of the ingredients 5 to 12 were homogenously dispersed with a Henschel mixer to produce a mixture II. The dispersion I and the mixture II were blended to prepare a cosmetic base slurry. The cosmetic base slurry was filled into a metal dish and was compacted under the following pressing conditions to produce a cheek cosmetic.

Pressing conditions: pressed twice for 2 sec at a load of 3 kgf/cm²,

<Evaluation>

The resulting cheek cosmetic was likewise evaluated as above to yield “⊙” in all of the formability, elasticity, uniform finger scooping, spreadability, tight adhesion, less pearl irregularity, moist finish, and impact resistance.

(Example 21: Eye Shadow)

| | (Blending ratio |
|---|---|
| (Ingredient) | (mass %)) |
| 1. Silicone wax A1 | 7.00 |
| 2. Silicone elastomer B2 | 20.00 |
| 3. Hydrogenated polydecene | 10.0 |
| 4. Hydrogenated (styrene/isoprene) copolymer | 2.0 |
| 5. Diisostearyl malate | 3.5 |
| 6. PEG-10 dimethicone | 1.0 |
| 7. Tocopherol | 0.01 |
| 8. Talc | 21.41 |
| 9. Mica titanium | 25.0 |
| 10. Titanium dioxide-coated glass flake | 10.0 |
| 11. Red iron oxide | 0.02 |
| 12. Yellow iron oxide | 0.01 |
| 13. Black iron oxide | 0.05 |

<Production Process>

The ingredients 1 to 7 were homogenously dispersed at 50° C. to produce a dispersion I. The powder ingredients of the ingredients 8 to 13 were homogenously dispersed with a Henschel mixer to produce a mixture II. The dispersion I and the mixture II were blended and further kneaded with 20 parts of light liquid isoparaffin as a dispersion medium to adjust a slurry. This slurry was filled into a metal dish, compacted under the following pressing conditions, and then dried to produce an eye shadow.

Pressing conditions: pressed twice for 2 sec at a load of 3 kgf/cm².

<Evaluation>

The resulting eye shadow was likewise evaluated as above to yield “⊙” in all of the formability, elasticity, uniform finger scooping, spreadability, tight adhesion, less pearl irregularity, moist finish, and impact resistance.

1. An oily solid cosmetic comprising;
a powder ingredient in an amount of 40 to 70 mass %, the powder ingredient comprising sparkling powder;
(A) silicone wax with a melting point of 25 to 55° C.; and
(B) a crosslinked silicone elastomer.

2. The oily solid cosmetic according to claim 1,
wherein content of the sparkling powder is 30 mass % or higher with reference to a total amount of the oily solid cosmetic.

3. The oily solid cosmetic according to claim 1,
wherein the silicone wax included has a melting point of 45° C. or lower and a hardness at 25° C. of 0.5 N to 5.0 N as measured with a rheometer under measurement conditions in which a tip shape of a pressure-sensing shaft is like a disk with a diameter of 5 mm, a penetration speed is 2 cm/min, and a penetration depth is 3 mm.

4. The oily solid cosmetic according to claim 1,
wherein a mixture comprising low-viscosity oil with a viscosity at 25° C. of 5 to 50 mPas and the crosslinked silicone elastomer dispersed in the low-viscosity oil is blended in an amount of 20 to 45 mass % with reference to a total amount of the oily solid cosmetic, and
the mixture has a hardness at 25° C. of 0.05 to 0.5 N as measured with a rheometer under measurement conditions in which a tip shape of a pressure-sensing shaft is like a sphere with a diameter of 10 mm, a penetration speed is 6 cm/min, and a penetration depth is 10 mm.

5. The oily solid cosmetic according to claim 1,
wherein the silicone wax (A) with a melting point of 25 to 55° C. is alkyl-modified silicone.

6. The oily solid cosmetic according to claim 1,
wherein the silicone wax (A) with a melting point of 25 to 55° C. is alkyl (C₂₆₋₂₈) dimethicone.

7. The oily solid cosmetic according to claim 4,
wherein the total content of the silicone wax (A) and the mixture containing the crosslinked silicone elastomer (B) in the oily solid cosmetic is from 30 mass % to 55 mass % with reference to the total amount of the oily solid cosmetic.

8. The oily solid cosmetic according to claim 1, which is a pressed molded product.

9. An oily solid cosmetic comprising:
a powder ingredient in an amount of 40 to 70 mass %, the powder ingredient comprising sparkling powder;
(A) silicone wax with a melting point of 25 to 55° C.; and
(B) a crosslinked silicone elastomer,
wherein a mixture comprising low-viscosity oil with a viscosity at 25° C. of 5 to 50 mPa·s and the crosslinked silicone elastomer dispersed in the low-viscosity oil is blended in an amount of 20 to 45 mass % with reference to a total amount of the oily solid cosmetic, and
wherein the silicone wax (A) with a melting point of 25 to 55° C. is alkyl-modified silicone.

10. The oily solid cosmetic according to claim 9,
wherein content of the sparkling powder is 30 mass % or higher with reference to a total amount of the oily solid cosmetic.

11. The oily solid cosmetic according to claim 9,
wherein the total content of the silicone wax (A) and the mixture containing the crosslinked silicone elastomer (B) in the oily solid cosmetic is from 30 mass % to 55 mass % with reference to the total amount of the oily solid cosmetic.

12. The oily solid cosmetic according to claim 9, which is a pressed molded product.
